(19) United States
(12) Patent Application Publication
(10) Pub. No.: US 2011/0178326 A1
(43) Pub. Date: Jul. 21, 2011

(54) UNSATURATED CINACALCET SALTS AND PROCESSES FOR PREPARING CINACALCET HYDROCHLORIDE

(75) Inventors: Sonny Sebastian, Kerala (IN); Seetha Rama Sarma, Hyderabad (IN); Katikireddy Ramamurthy, Andhra Pradesh (IN); Nitin Sharadchandra Pradhan, Maharashtra (IN)

(73) Assignee: ACTAVIS GROUP PTC EHF, Hafnarfjordur (IS)

(21) Appl. No.: 13/057,796

(22) PCT Filed: Aug. 6, 2009
(86) PCT No.: PCT/IB09/06708

§ 371 Date: Apr. 4, 2011

(30) Foreign Application Priority Data
Aug. 6, 2008 (IN) 1887/CHE/2008

(51) Int. Cl. C07C 271/14; C07C 209/70; C07C 209/68; C07C 211/30; C07C 209/86

(52) U.S. Cl. 560/28; 564/373

(57) ABSTRACT

Disclosed herein are convenient, industrially advantageous and environmentally friendly processes for the preparation of cinacalcet hydrochloride. Disclosed also herein are novel hydrochloride, oxalate and di-p-toluoyl-L-(+)-tartrate salts of (R)-α-methyl-N-[3-[3-(trifluoromethyl)phenyl]propylene]-1-naphthalenemethaneamine (unsaturated cinacalcet), solid state forms of the salts, and a process for their preparation thereof.

### CROSS REFERENCE TO RELATED APPLICATION

This application claims the benefit of priority to Indian provisional application No. 1887/CHE/2008, filed on Aug. 6, 2008, which is incorporated herein by reference in its entirety.

### FIELD OF THE DISCLOSURE

The present disclosure relates to processes for the preparation of cinacalcet hydrochloride. The present disclosure further relates to novel salts of (R)-α-methyl-N-[3-[3-(trifluoromethyl)phenyl]propylene]-1-naphthalenemethaneamine, solid state forms of the salts, and a process for their preparation thereof.

### BACKGROUND

Cinacalcet, chemically known as (R)-α-methyl-N-[3-[3-(trifluoromethyl)phenyl]propyl]-1-naphthalenemethane amine, is an important antihyperparathyroic agent and acts as a calcimimetic by allostric activation of the calcium sensing receptor that is expressed in various human organ tissues. Cinacalcet is used to treat secondary hyperparathyroidism in patients with chronic kidney disease and hypercalcemia in patients with parathyroid carcinoma. Cinacalcet hydrochloride is sold by Amgen under the trade name SENSIPAR™ in the USA and as MIMPARA™ in Europe. Cinacalcet hydrochloride is represented by the following structural formula I:

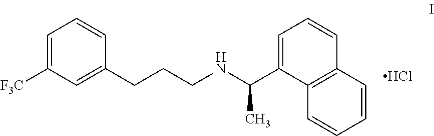

U.S. Pat. No. 6,011,068 generally describes cinacalcet and its pharmaceutically acceptable acid addition salts thereof.

U.S. Pat. No. 6,211,244 describes cinacalcet and related compounds, and their pharmaceutically acceptable salts thereof.

Processes for the preparation of cinacalcet and related compounds, and their pharmaceutically acceptable salts are disclosed in U.S. Pat. Nos. 6,211,244; 7,250,533; 5,648,541; 7,247,751; and 7,393,967; PCT Publication Nos. WO06/127933; WO06/125026; WO06/127941; WO07/062,147; WO07/112,280; WO07/127,445; WO07/127,449; WO08/058,235; WO08/000,423; WO08/035,212; WO08/058,236; WO08/063,645; and WO08/068,625.

According to U.S. Pat. No. 6,211,244, cinacalcet or its analogues are prepared by the reaction of 3-[(3-trifluoromethyl)phenyl]cinnamaldehyde or a derivative thereof with R-(+)-1-(1-naphthyl)ethyl amine or a derivative thereof in the presence of titanium(IV) isopropoxide. The resulting intermediate imines are reduced in situ by the action of sodiumcyanoborohydride, sodiumborohydride or sodium triacetoxyborohydride. The intermediate enamine is catalytically reduced using palladium or palladium hydroxide on carbon to produce cinacalcet base or its analogues. Hydrochlorides of these analogues are prepared by the precipitation using gaseous HCl in ether or hexane in combination with gaseous HCl in ether.

A need still remains for improved and commercially viable processes of preparing pure cinacalcet hydrochloride.

### SUMMARY

The hydrochloride, oxalate and di-p-toluoyl-L-(+)-tartrate salts of unsaturated cinacalcet, have not been reported, isolated, or characterized in the literature. The present inventors have surprisingly and unexpectedly found that hydrochloride, oxalate and di-p-toluoyl-L-(+)-tartrate salts of (R)-α-methyl-N-[3-[3-(trifluoromethyl)phenyl]propylene]-1-naphthalenemethaneamine, i.e., unsaturated cinacalcet hydrochloride, unsaturated cinacalcet oxalate and unsaturated cinacalcet di-p-toluoyl-L-(+)-tartrate salts, can be isolated in a pure solid state form.

In one aspect, provided herein are novel hydrochloride, oxalate and di-p-toluoyl-L-(+)-tartrate salts of unsaturated cinacalcet. In another aspect, unsaturated cinacalcet hydrochloride, oxalate and di-p-toluoyl-L-(+)-tartrate salts in a solid state form are provided.

In another aspect, unsaturated cinacalcet salts in a crystalline form are provided. In yet another aspect, unsaturated cinacalcet salts in an amorphous form are provided. In still another aspect, the solid state forms of unsaturated cinacalcet salts exist in an anhydrous and/or solvent-free form or as a hydrate and/or a solvate.

It has also been found that the novel hydrochloride, oxalate and di-p-toluoyl-L-(+)-tartrate salts of unsaturated cinacalcet are useful intermediates in the preparation of cinacalcet free base or a pharmaceutically acceptable salt thereof, preferably cinacalcet hydrochloride, in high purity.

In another aspect, encompassed herein is a process for preparing the novel hydrochloride, oxalate and di-p-toluoyl-L-(+)-tartrate salts of unsaturated cinacalcet comprising contacting unsaturated cinacalcet free base with an acid counter ion in a suitable solvent, and isolating the solid state form of unsaturated cinacalcet acid addition salt, wherein the acid counter ion is provided by an acid selected from the group consisting of hydrochloric acid, oxalic acid and di-p-toluoyl-L-(+)-tartaric acid.

In another aspect, encompassed herein is a process for preparing the highly pure cinacalcet hydrochloride by using the solid state forms of unsaturated cinacalcet hydrochloride, oxalate and di-p-toluoyl-L-(+)-tartrate salts disclosed herein.

In another aspect, provided herein are efficient, industrially advantageous and environment friendly processes for the preparation of cinacalcet hydrochloride.

### BRIEF DESCRIPTION OF THE DRAWINGS

FIG. 1 is a characteristic powder X-ray diffraction (XRD) pattern of unsaturated cinacalcet hydrochloride salt.

FIG. 2 is a characteristic infra red (IR) spectrum of unsaturated cinacalcet hydrochloride salt.

FIG. 3 is a characteristic powder X-ray diffraction (XRD) pattern of unsaturated cinacalcet oxalate salt.

FIG. 4 is a characteristic infra red (IR) spectrum of unsaturated cinacalcet oxalate salt.

FIG. 5 is a characteristic powder X-ray diffraction (XRD) pattern of unsaturated cinacalcet di-p-toluoyl-L-(+)-tartrate salt.

FIG. 6 is a characteristic infra red (IR) spectrum of unsaturated cinacalcet di-p-toluoyl-L-(+)-tartrate salt.

### DETAILED DESCRIPTION

According to one aspect, there is provided a process for the preparation of cinacalcet hydrochloride of formula I:

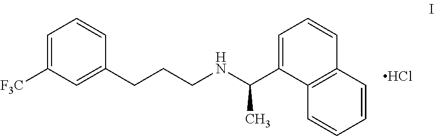

comprising hydrogenating (R)-α-methyl-N-[3-[3-(trifluoromethyl)phenyl]propylene]-1-naphthalenemethaneamine hydrochloride salt (unsaturated cinacalcet hydrochloride) of formula III:

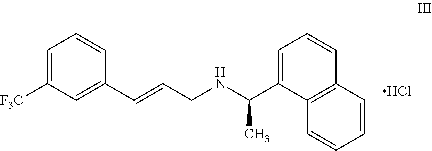

in the presence of a hydrogenation catalyst in a solvent to provide substantially pure cinacalcet hydrochloride of formula I.

The term “substantially pure cinacalcet hydrochloride” refers to cinacalcet hydrochloride having a total purity of greater than about 99%, specifically greater than about 99.5%, more specifically greater than about 99.9%, and still more specifically greater than about 99.95%. The purity is preferably measured by High Performance Liquid Chromatography (HPLC). For example, the purity of cinacalcet hydrochloride obtained by the process disclosed herein is about 99% to about 99.95%, or about 99.5% to about 99.99%, as measured by HPLC.

Exemplary hydrogenation catalysts include, but are not limited to, palladium hydroxide, palladium on carbon, platinum on carbon, platinum oxide, rhodium on carbon, rhodium on alumina. A specific hydrogenation catalyst is palladium hydroxide.

Exemplary solvents used for the hydrogenation include, but are not limited to, water, an alcohol, a ketone, an ester, acetonitrile, tetrahydrofuran, dimethylformamide, dimethylsulfoxide, dioxane, diethyl carbonate, and mixtures thereof. The term solvent also includes mixtures of solvents.

In one embodiment, the solvent is selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; more specifically, the solvent is selected from the group consisting of methanol, ethanol, isopropyl alcohol, n-butanol, and mixtures thereof; and most specifically, the solvent is selected from the group consisting of methanol, n-butanol, and mixtures thereof.

In one embodiment, the hydrogenation reaction is carried out at a temperature of below about 50° C. for at least 30 minutes, specifically at a temperature of about −25° C. to about 40° C. for about 1 hour to about 7 hours, and more specifically at about 0° C. to about 20° C. for about 2 hours to about 5 hours.

In another embodiment, the hydrogenation catalyst is used in the ratio of about 0.05% (w/w) to 10% (w/w), specifically about 0.5% (w/w) to 2.5% (w/w), with respect to the unsaturated cinacalcet hydrochloride in order to ensure a proper course of the reaction.

The reaction mass containing the cinacalcet hydrochloride of formula I obtained after hydrogenation may be subjected to usual work up such as a washing, an extraction, an evaporation or a combination thereof, followed by isolation as solid from a suitable organic solvent by conventional methods such as cooling, seeding, partial removal of the solvent from the solution, by adding an anti-solvent to the solution, evaporation, vacuum drying, spray drying, freeze drying, or a combination thereof.

(R)-α-Methyl-N-[3-[3-(trifluoromethyl)phenyl]propylene]-1-naphthalenemethane amine hydrochloride salt (unsaturated cinacalcet hydrochloride) of formula III used as starting material is prepared by the process disclosed hereinafter.

According to another aspect, there is provided a process for the preparation of cinacalcet hydrochloride of formula I, comprising:
a) reacting cinacalcet free base of formula II:

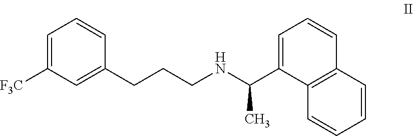

with a nitrogen protecting agent in the presence of a base in a first solvent to provide N-protected cinacalcet of formula IV:

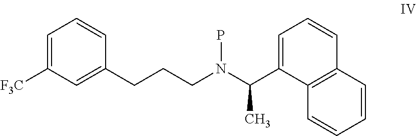

wherein ‘P’ represents a nitrogen protecting group; and
b) converting the compound of formula IV into substantially pure cinacalcet hydrochloride of formula I by reaction with hydrochloric acid in a second solvent.

Exemplary first solvents used in step-(a) include, but are not limited to, water, an alcohol, a ketone, an ester, acetonitrile, tetrahydrofuran, dimethylformamide, dimethylsulfoxide, dioxane, diethyl carbonate, and mixtures thereof. The term solvent also includes mixtures of solvents.

Specifically, the first solvent is selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; more specifically, the first solvent is selected from the group consisting of water, tetrahydrofuran, dimethylformamide, dimethylsulfoxide, and mixtures thereof; and most specifically, the first solvent is selected from the group consisting of water, tetrahydrofuran, and mixtures thereof.

In one embodiment, the base used in step-(a) is an organic or inorganic base. Exemplary organic bases are triethyl amine, tributylamine, diisopropylethylamine, diethylamine, tert-butyl amine, N-methylmorpholine, pyridine, and 4-(N,N-dimethylamino)pyridine. Exemplary inorganic bases include, but are not limited to, hydroxides, carbonates and bicarbonates of alkali or alkaline earth metals. Specific inorganic bases are sodium hydroxide, calcium hydroxide, magnesium hydroxide, potassium hydroxide, lithium hydroxide, sodium carbonate, potassium carbonate, lithium carbonate, sodium bicarbonate, potassium bicarbonate, and more specifically sodium bicarbonate, sodium hydroxide, potassium hydroxide, sodium carbonate and potassium carbonate.

Exemplary nitrogen protecting agents are conventionally used in peptide chemistry and are described e.g. in the relevant chapters of standard reference works such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981.

In one embodiment, the nitrogen protecting agent is an amine protecting agent selected from the group consisting of an acid anhydride, a mixed anhydride, an acid chloride, an alkyl halide, an aralkyl halide and a silyl compound. A specific nitrogen protecting agent is di-tert-butyl-dicarbonate.

In another embodiment, the nitrogen protecting agent is used in the molar ratio of about 1 to 5 moles, specifically about 1 to 2 moles, per 1 mole of the cinacalcet free base of formula II in order to ensure a proper course of the reaction.

In one embodiment, the reaction in step-(a) is carried out at a temperature of below the boiling temperature of the solvent used, specifically at a temperature of about 0° C. to about 60° C. for at least 1 hour, and more specifically at about 10° C. to about 40° C. for about 5 hours to about 15 hours. In another embodiment, the reaction mass may be quenched with water after completion of the reaction.

Exemplary nitrogen protecting groups ‘P’ include, but are not limited to, acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl. Specific nitrogen protecting groups are acetyl, benzyloxycarbonyl (Cbz), trimethylsilyl, triethylsilyl (TES), trimethylsilyethoxymethyl (SEM), tert-butoxycarbonyl (BOC) and pivaloyl. A most specific nitrogen protecting group is tert-butoxycarbonyl (BOC).

The reaction mass containing the compound of formula IV obtained in step-(a) may be subjected to usual work up such as a washing, an extraction, an evaporation or a combination thereof. The reaction mass may be used directly in the next step to produce cinacalcet hydrochloride of formula I, or the compound of formula IV may be isolated by the methods described hereinabove and then used in the next step.

The compounds of formula IV are novel and constitute another aspect of the disclosure.

In one embodiment, a specific N-protected compound of formula IV prepared by the process described herein is N—BOC protected cinacalcet of formula IV(i) (formula IV, wherein P is tert-butoxycarbonyl):

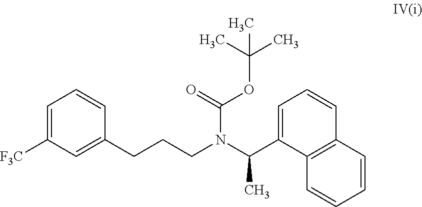

Exemplary second solvents used in step-(b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the second solvent is selected from the group consisting of water, methanol, ethanol, isopropyl alcohol, n-butanol, and mixtures thereof; and most specifically, the second solvent is selected from the group consisting of water, methanol, n-butanol, and mixtures thereof.

The hydrochloric acid used may be in the form of concentrated hydrochloric acid or aqueous hydrochloric acid or in the form of hydrogen chloride gas or hydrogen chloride dissolved in an organic solvent. In one embodiment, the organic solvent is selected from the group consisting of ethanol, methanol, isopropyl alcohol, ethyl acetate, diethyl ether, dimethyl ether and acetone.

In one embodiment, the reaction in step-(b) is carried out at a temperature of about −25° C. to the reflux temperature of the solvent used, specifically at a temperature of about 0° C. to the reflux temperature of the solvent, more specifically at about 25° C. to the reflux temperature of the solvent, and most specifically at the reflux temperature of the solvent.

As used herein, “reflux temperature” means the temperature at which the solvent or solvent system refluxes or boils at atmospheric pressure.

The reaction mass containing the cinacalcet hydrochloride of formula I obtained may be subjected to usual work up such as a filtration, a washing, an extractions, an evaporation, or a combination thereof, followed by isolation as a solid from a suitable organic solvent by the methods described hereinabove.

Cinacalcet free base of formula II used as starting material may be obtained by processes described in the prior art, or by the process disclosed herein.

According to another aspect, there is provided a process for the preparation of cinacalcet hydrochloride of formula I, comprising:
a) neutralizing (R)-α-methyl-N-[3-[3-(trifluoromethyl)phenyl]propylene]-1-naphthalene methaneamine hydrochloride salt of formula III:

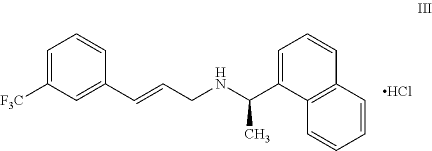

with a base in a first solvent to provide (R)-α-methyl-N-[3-[3-(trifluoromethyl)phenyl]propylene]-1-naphthalene methaneamine of formula V:

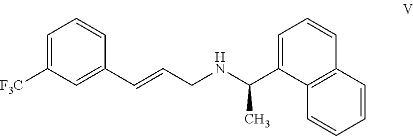

b) reacting the unsaturated compound of formula V with a nitrogen protecting agent in the presence of a base in a second solvent to provide N-protected unsaturated compound of formula VI:

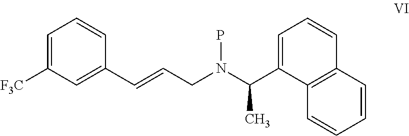

wherein ‘P’ represents a nitrogen protecting group;
c) hydrogenating the compound of formula VI in the presence of a hydrogenation catalyst in a third solvent to provide the N-protected cinacalcet of formula IV:

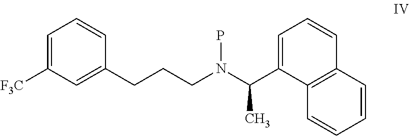

wherein P is as defined in formula VI;
d) converting the compound of formula IV into substantially pure cinacalcet hydrochloride of formula I by reaction with hydrochloric acid in a fourth solvent.

Exemplary first solvents used in step-(a) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the first solvent is selected from the group consisting of water, ethyl acetate, methyl acetate, isopropyl acetate, tert-butyl methyl acetate, ethyl formate, and mixtures thereof; and most specifically, the first solvent is selected from the group consisting of water, ethyl acetate, and mixtures thereof.

In one embodiment, the base used in step-(a) is an organic or inorganic base selected from the group as described above.

The reaction mass containing the compound of formula V obtained in step-(a) may be subjected to usual work up such as a washing, an extraction, an evaporation, or a combination thereof. The reaction mass may be used directly in the next step to produce N—BOC protected unsaturated compound of formula VI, or the compound of formula V may be isolated by the methods described hereinabove and then used in the next step.

Exemplary second solvents used in step-(b) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the second solvent is selected from the group consisting water, tetrahydrofuran, dimethylformamide, dimethylsulfoxide, and mixtures thereof; and most specifically, the second solvent is selected from the group consisting of water, tetrahydrofuran, and mixtures thereof.

In one embodiment, the base used in step-(b) is an organic or inorganic base selected from the group as described above.

In one embodiment, the nitrogen protecting agent is an amine protecting agent selected from the group as described above. A specific nitrogen protecting agent is di-tert-butyl-dicarbonate.

In another embodiment, the nitrogen protecting agent is used in the molar ratio of about 1 to 5 moles, specifically about 1 to 2 moles, per 1 mole of the (R)-α-methyl-N-[3-[3-(trifluoromethyl)phenyl]propylene]-1-naphthalenemethaneamine of formula V in order to ensure a proper course of the reaction.

In one embodiment, the reaction in step-(b) is carried out at a temperature of below the boiling temperature of the solvent used, specifically at a temperature of about 0° C. to about 60° C. for at least 1 hour, and more specifically at about 10° C. to about 40° C. for about 5 hours to about 15 hours. In another embodiment, the reaction mass may be quenched with water after completion of the reaction.

In another embodiment, the nitrogen protecting group ‘P’ is selected from the group as described above. Specific nitrogen protecting groups are acetyl, benzyloxycarbonyl (Cbz), trimethylsilyl, triethylsilyl (TES), trimethylsilyethoxymethyl (SEM), tert-butoxycarbonyl (BOC) and pivaloyl; and a most specific nitrogen protecting group is tert-butoxycarbonyl (BOC).

The reaction mass containing the N-protected unsaturated compound of formula VI obtained in step-(b) may be subjected to usual work up such as a washing, an extraction, an evaporation, or a combination thereof. The reaction mass may be used directly in the next step to produce N-protected cinacalcet of formula IV, or the compound of formula VI may be isolated by the methods described hereinabove and then used in the next step.

The compounds of formula VI are novel and constitute another aspect of the invention.

In one embodiment, a specific N-protected unsaturated compound of formula VI prepared by the process described herein is the N—BOC protected unsaturated cinacalcet of formula VI(i) (formula VI, wherein P is tert-butoxycarbonyl):

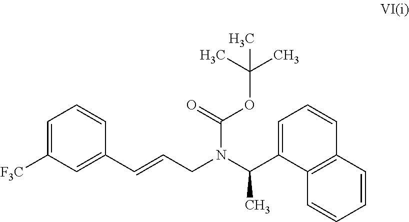

In another embodiment, the hydrogenation catalyst used in step-(c) is selected from the group as described above. A specific hydrogenation catalyst is palladium hydroxide.

Exemplary third solvents used in step-(c) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the third solvent is selected from the group consisting methanol, ethanol, isopropyl alcohol, n-butanol, and mixtures thereof; and most specifically, the third solvent is selected from the group consisting of methanol, n-butanol, and mixtures thereof.

In one embodiment, the hydrogenation reaction in step-(c) is carried out at a temperature of below about 50° C. for at least 30 minutes, specifically at a temperature of about −25° C. to about 40° C. for about 1 hour to about 7 hours, and more specifically at about 0° C. to about 20° C. for about 2 hours to about 5 hours.

In another embodiment, the hydrogenation catalyst is used in the ratio of about 0.05% (w/w) to 10% (w/w), specifically about 0.5% (w/w) to 2.5% (w/w), with respect to the compound of formula VI in order to ensure a proper course of the reaction.

The reaction mass containing N-protected cinacalcet of formula IV obtained in step-(c) may be subjected to usual work up such as a filtration, a washing, an extraction, an evaporation, or a combination thereof. The reaction mass may be used directly in the next step to produce cinacalcet hydrochloride of formula I, or the compound of formula IV may be isolated by the methods described hereinabove and then used in the next step.

Exemplary fourth solvents used in step-(d) include, but are not limited to, water, an alcohol, a ketone, an ester, acetonitrile, tetrahydrofuran, dimethylformamide, dimethylsulfoxide, dioxane, diethyl carbonate, and mixtures thereof. Specifically, the fourth solvent is selected from the group consisting water, methanol, ethanol, isopropyl alcohol, n-butanol, and mixtures thereof; and most specifically, the fourth solvent is selected from the group consisting of water, methanol, n-butanol, and mixtures thereof.

The hydrochloric acid used may be in the form of concentrated hydrochloric acid or aqueous hydrochloric acid or in the form of hydrogen chloride gas or hydrogen chloride dissolved in an organic solvent as described above.

In one embodiment, the reaction in step-(d) is carried out at a temperature of −25° C. to the reflux temperature of the solvent used, specifically at a temperature of 0° C. to the reflux temperature of the solvent, more specifically at a temperature of 25° C. to the reflux temperature of the solvent, and most specifically at the reflux temperature of the solvent.

The reaction mass containing the cinacalcet hydrochloride of formula I obtained may be subjected to usual work up such as a filtration, a washing, an extractions, an evaporation, or a combination thereof, followed by isolation as a solid from a suitable organic solvent by the methods described hereinabove.

(R)-α-Methyl-N-[3-[3-(trifluoromethyl)phenyl]propylene]-1-naphthalenemethane amine hydrochloride salt (unsaturated cinacalcet hydrochloride) of formula III used as starting material may be obtained by the process disclosed herein.

The total purity of the cinacalcet hydrochloride obtained by the processes disclosed herein is greater than about 99.7%, specifically greater than about 99.90%, and more specifically greater than about 99.95% as measured by HPLC.

The highly pure cinacalcet hydrochloride obtained by the above processes may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, provided herein are novel hydrochloride, oxalate and di-p-toluoyl-L-(+)-tartrate salts of (R)-α-methyl-N-[3-[3-(trifluoromethyl)phenyl]propylene]-1-naphthalene methane amine (unsaturated cinacalcet).

According to another aspect, provided herein are solid state forms of unsaturated cinacalcet salts, wherein the salt of unsaturated cinacalcet is a hydrochloride salt, an oxalate salt or a di-p-toluoyl-L-(+)-tartrate salt.

In one embodiment, the solid state forms of unsaturated cinacalcet hydrochloride, oxalate and di-p-toluoyl-L-(+)-tartrate salts exist in a crystalline form. In another embodiment, the solid state forms of unsaturated cinacalcet hydrochloride, oxalate and di-p-toluoyl-L-(+)-tartrate salts exist in an amorphous form.

In yet another embodiment, the solid state forms of unsaturated cinacalcet hydrochloride, oxalate and di-p-toluoyl-L-(+)-tartrate salts exist in an anhydrous and/or solvent-free form or as a hydrate and/or a solvate form. Such solvated or hydrated forms may be present as hemi-, mono-, sesqui-, di- or tri-solvates or hydrates. Solvates and hydrates may be formed as a result of solvents used during the formation of the unsaturated cinacalcet salts becoming imbedded in the solid lattice structure. Because formation of the solvates and hydrates occurs during the preparation of unsaturated cinacalcet salts, formation of a particular solvated or hydrated form depends greatly on the conditions and method used to prepare the salt. Solvents should be pharmaceutically acceptable.

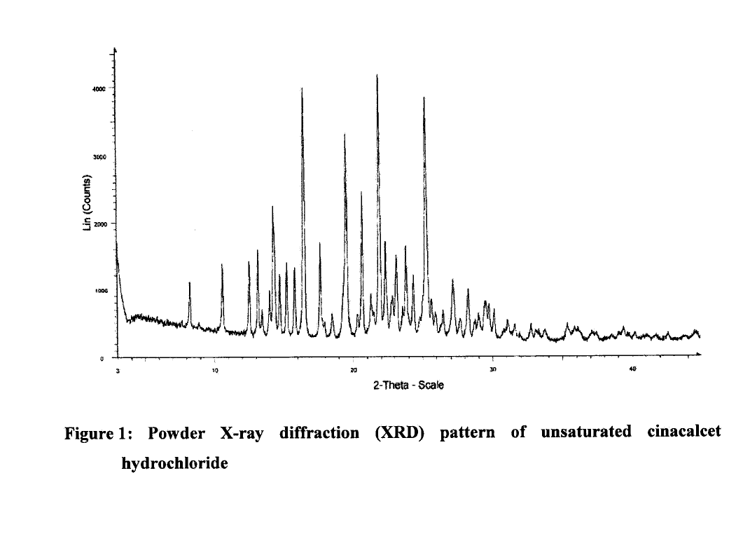

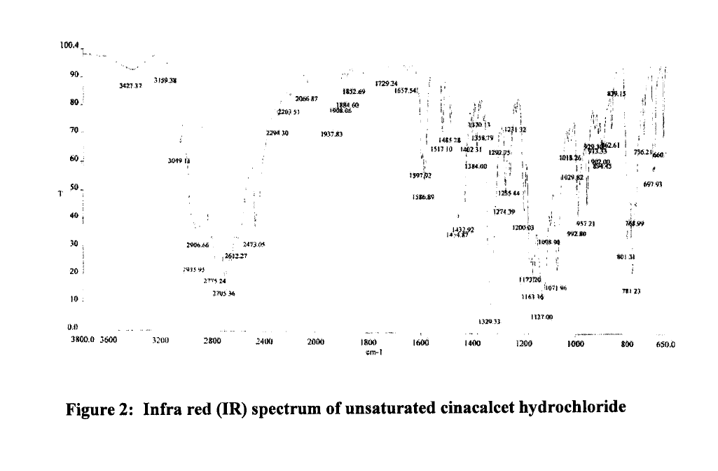

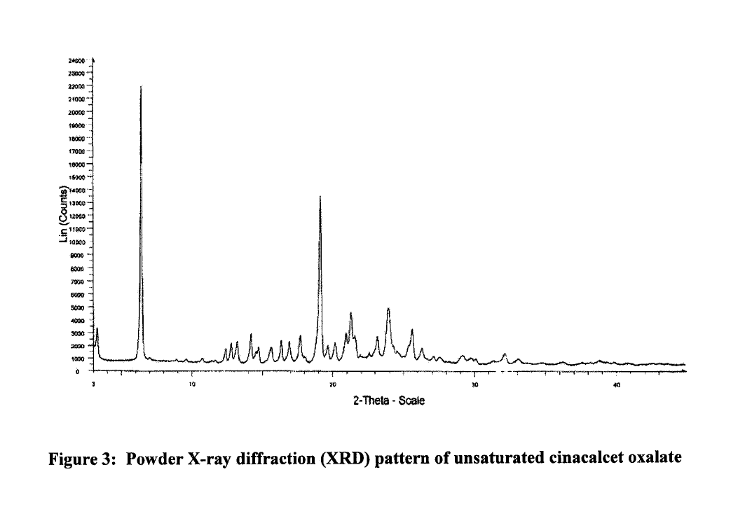

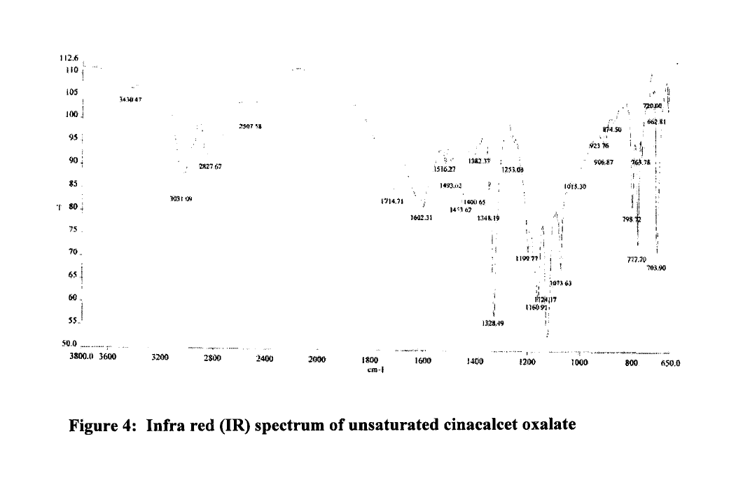

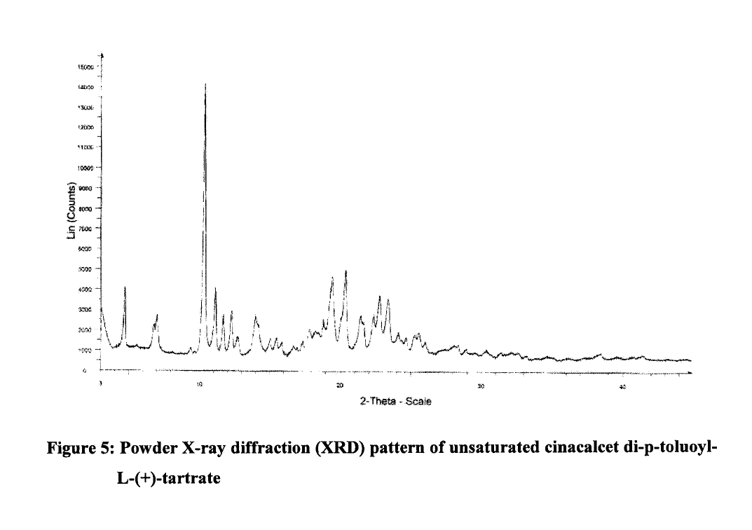

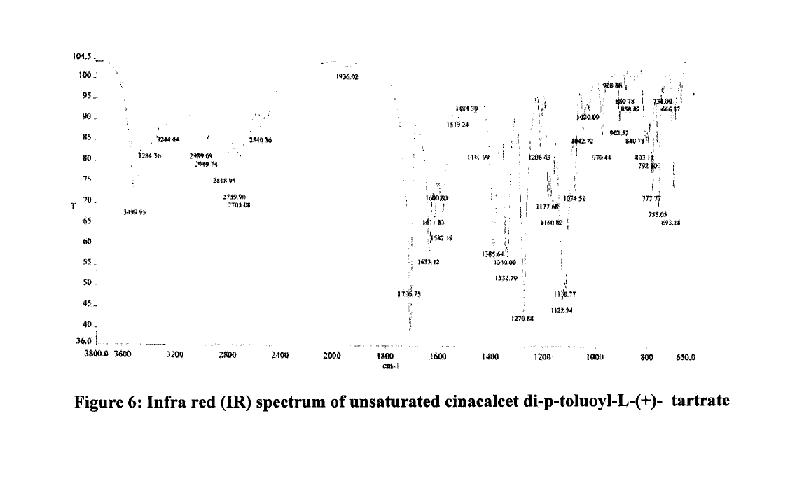

In one embodiment, the solid state forms of unsaturated cinacalcet salts have the following characteristics, wherein:
a) the solid state form of unsaturated cinacalcet hydrochloride salt is characterized by one or more of the following properties:
   i) a powder X-ray diffraction pattern substantially in accordance with FIG. 1;
   ii) a powder X-ray diffraction pattern having peaks at about 8.2, 14.3, 16.5, 19.5 and 21.9±0.2 degrees 2-theta;
   iii) a powder X-ray diffraction pattern having additional peaks at about 10.5, 12.5, 13.1, 13.9, 14.7, 15.2, 15.8, 17.6, 20.6, 22.3, 23.1, 23.8, 24.3, 27.2 and 28.2±0.2 degrees 2-theta;
   iv) an IR spectrum substantially in accordance with FIG. 2; and
   v) an IR spectrum having absorption bands at 3427, 3049, 2935, 2906, 2775, 2705, 2473, 1597, 1586, 1454, 1432, 1384, 1329, 1274, 1255, 1200, 1173, 1163, 1127, 1071, 992, 957, 801, 781, 768 and 697±1 cm⁻¹;
b) the solid state form of unsaturated cinacalcet oxalate salt is characterized by one or more of the following properties:
   i) a powder X-ray diffraction pattern substantially in accordance with FIG. 3;
   ii) a powder X-ray diffraction pattern having peaks at about 6.3, 19.1 and 23.9±0.2 degrees 2-theta;
   iii) a powder X-ray diffraction pattern having additional peaks at about 3.2, 12.7, 13.2, 14.1, 16.3, 16.9, 17.6, 20.2, 20.9, 21.3, 21.5, 23.1 and 25.6±0.2 degrees 2-theta;
   iv) an IR spectrum substantially in accordance with FIG. 4; and
   v) an IR spectrum having absorption bands at 3430, 3031, 2827, 1714, 1602, 1453, 1400, 1348, 1328, 1199, 1160, 1124, 1073, 906, 798, 777 and 703±1 cm⁻¹; and
c) the solid state form of unsaturated cinacalcet di-p-toluoyl-L-(+)-tartrate salt is characterized by one or more of the following properties:
   i) a powder X-ray diffraction pattern substantially in accordance with FIG. 5;
   ii) a powder X-ray diffraction pattern having peaks at about 4.6, 10.3, 11.1, 19.4, 20.3, 22.8 and 23.4±0.2 degrees 2-theta;
   iii) a powder X-ray diffraction pattern having additional peaks at about 6.7, 6.9, 11.7, 12.3, 12.7, 13.9, 17.8, 18.4, 18.8, 20.0, 21.4, 22.3 and 25.6±0.2 degrees 2-theta;
   iv) an IR spectrum substantially in accordance with FIG. 6; and
   v) an IR spectrum having absorption bands at 3499, 3384, 2989, 2949, 2818, 2739, 2705, 1706, 1633, 1611, 1582, 1440, 1385, 1340, 1332, 1270, 1177, 1160, 1122, 1110, 1074, 970, 902, 777, 755 and 695±1 cm⁻¹.

The solid state forms of unsaturated cinacalcet hydrochloride, oxalate and di-p-toluoyl-L-(+)-tartrate salts are stable, consistently reproducible, and are particularly suitable for bulk preparation and handling. Moreover, the solid state forms of unsaturated cinacalcet hydrochloride, oxalate and di-p-toluoyl-L-(+)-tartrate salts are useful intermediates in the preparation of cinacalcet or a pharmaceutically acceptable salt thereof, specifically cinacalcet hydrochloride, in high purity.

According to another aspect, there is provided a process for the preparation of an unsaturated cinacalcet salt, wherein the salt is a hydrochloride, an oxalate or a dip-toluoyl-L-(+)-tartrate, comprising:
a) providing a first solution of unsaturated cinacalcet free base in a solvent;
b) combining the first solution with an acid to produce a second solution or suspension containing unsaturated cinacalcet acid addition salt, wherein the acid is selected from the group consisting of hydrochloric acid, oxalic acid and di-p-toluoyl-L-(+)-tartaric acid; and
c) isolating and/or recovering solid state form of unsaturated cinacalcet salt from the second solution or suspension.

The solid state form of unsaturated cinacalcet salt obtained by the process disclosed herein is optionally converted into cinacalcet free base or a pharmaceutically acceptable salt thereof, specifically cinacalcet hydrochloride.

The process can produce solid state forms of unsaturated cinacalcet hydrochloride, oxalate and di-p-toluoyl-L-(+)-tartrate salts in substantially pure form.

The term “substantially pure solid state form of unsaturated cinacalcet salt” refers to the solid state form of unsaturated cinacalcet salt having a total purity of greater than about 98%, specifically greater than about 99%, more specifically greater than about 99.5%, and still more specifically greater than about 99.9%. The purity is preferably measured by High Performance Liquid Chromatography (HPLC). For example, the purity of solid state form of unsaturated cinacalcet salt obtained by the process disclosed herein can be about 98% to about 99.95%, or about 99.5% to about 99.99%, as measured by HPLC.

In one embodiment, the process disclosed herein provides stable crystalline forms of unsaturated cinacalcet hydrochloride, oxalate and di-p-toluoyl-L-(+)-tartrate salts. The term “stable crystalline form” refers to stability of the crystalline form under the standard temperature and humidity conditions of testing of pharmaceutical products, wherein the stability is indicated by preservation of the original polymorphic form.

Exemplary solvents used in step-(a) include, but are not limited to, water, an ether, an alcohol, a ketone, a chlorinated hydrocarbon, a hydrocarbon, an ester, a nitrile, and mixtures thereof. The term solvent also includes mixtures of solvents.

In one embodiment, the solvent is selected from the group consisting of water, methyl tert-butyl ether, diethyl ether, diisopropyl ether, monoglyme, diglyme, tetrahydrofuran, dioxane, methanol, ethanol, n-propanol, isopropyl alcohol, isobutanol, n-butanol, tert-butanol, amyl alcohol, isoamyl alcohol, acetone, methyl ethyl ketone, methyl isobutyl ketone, methyl tert-butyl ketone, ethyl acetate, methyl acetate, isopropyl acetate, tert-butyl methyl acetate, ethyl formate, methylene chloride, ethylene dichloride, n-pentane, n-hexane, n-heptane and their isomers, cyclohexane, toluene, xylene, acetonitrile, and mixtures thereof. Specifically the solvent is selected from the group consisting of water, acetonitrile, methanol, ethanol, n-propanol, isopropyl alcohol, isobutanol, n-butanol, tert-butanol, acetone, and mixtures thereof; and most specifically, the solvent is selected from the group consisting of water, methanol, ethanol, isopropyl alcohol, acetonitrile, and mixtures thereof.

Step-(a) of providing a first solution of unsaturated cinacalcet free base includes dissolving unsaturated cinacalcet free base in the solvent, or obtaining an existing solution from a previous processing step.

In one embodiment, the unsaturated cinacalcet free base is dissolved in the solvent at a temperature of 0° C. to the boiling temperature of the solvent used, specifically at about 20° C. to about 100° C., and more specifically at about 25° C. to about 80° C.

In another embodiment, the first solution in step-(a) is prepared by reacting 3-(trifluoromethyl)cinnamaldehyde with (R)-(+)-1-(1-naphthyl)ethyl amine or an acid addition salt thereof in the presence of a reducing agent in a solvent to produce a reaction mass containing (R)-α-methyl-N-[3-[3-(trifluoromethyl)phenyl]propylene]-1-naphthalenemethane-amine (unsaturated cinacalcet free base), followed by usual work up such as a filtration, a washing, an extraction, an evaporations, or a combination thereof. In one embodiment, the work-up includes dissolving or extracting the resulting unsaturated cinacalcet free base in the solvent at a temperature of 0° C. to the boiling temperature of the solvent used, specifically at about 20° C. to about 100° C., and more specifically at about 25° C. to about 80° C.

Exemplary reducing agents suitable for facilitating the reaction between 3-(trifluoromethyl)cinnamaldehyde with (R)-(+)-1-(1-naphthyl)ethyl amine include, but are not limited to, sodium borohydride, sodium cyanoborohydride, and sodium triacetoxyborohydride. A specific reducing agent is sodium borohydride. In one embodiment, the reducing agent is used in a molar ratio of about 0.5 to 3.0 moles, specifically about 1.0 to 2.0 moles, per 1 mole of the 3-(trifluoromethyl)cinnamaldehyde.

Exemplary solvents used in the reaction between 3-(trifluoromethyl)cinnamaldehyde and (R)-(+)-1-(1-naphthyl)ethyl amine include, but are not limited to, an alcohol, a ketone an ester, acetonitrile, tetrahydrofuran, dimethylformamide, dimethylsulfoxide, dioxane; diethyl carbonate, and mixtures thereof. Specific solvents are methanol, ethanol, isopropyl alcohol, n-butanol and mixtures thereof; and most specifically, methanol, n-butanol, and mixtures thereof. In one embodiment, the reaction is carried out at a temperature of below boiling temperature of the solvent used, specifically at a temperature of about −25° C. to about 50° C. for at least 30 minutes, and most specifically at a temperature of about 15° C. to about 35° C. for about 2 hours to about 6 hours.

Alternatively, the first solution in step-(a) is prepared by treating an acid addition salt of unsaturated cinacalcet with a base to liberate unsaturated cinacalcet free base and dissolving or extracting the unsaturated cinacalcet free base in the solvent.

The acid addition salts are derived from a therapeutically acceptable acid such as hydrochloric acid, hydrobromic acid, sulfuric acid, nitric acid, oxalic acid, acetic acid, propionic acid and, phosphoric acid, succinic acid, maleic acid, fumaric acid, citric acid, glutaric acid, citraconic acid, glutaconic acid, tartaric acid, di-p-toluoyl-L-(+)-tartaric acid, malic acid, and ascorbic acid.

The treatment of an acid addition salt with base is carried out in a solvent selected from the group consisting of water, an ester, an alcohol, a ketone, a chlorinated hydrocarbon, a hydrocarbon, an ether, and mixtures thereof.

The base can be inorganic or organic base selected from the group as described hereinabove. A specific base is an inorganic base selected from alkali metal hydroxides, carbonates and bicarbonates.

The first solution obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing unsaturated cinacalcet free base by removing charcoal or silica gel. Preferably, finely powdered carbon is an active carbon. A specific mesh size of silica gel is 40-500 mesh, and more specifically 60-120 mesh.

In another embodiment, the acid used in step-(b) is in a molar ratio of about 1.0 to 2.0 moles, specifically about 1.0 to 1.2 moles, per mole of unsaturated cinacalcet free base.

In one embodiment, the hydrochloric acid used in step-(b) is in the form of a concentrated hydrochloric acid or an aqueous hydrochloric acid or in the form of hydrogen chloride gas or hydrogen chloride dissolved in an organic solvent. In another embodiment, the organic solvent used for dissolving hydrogen chloride gas or hydrogen chloride is selected from the group consisting of ethanol, methanol, isopropyl alcohol, ethyl acetate, diethyl ether, dimethyl ether and acetone.

In another embodiment, the oxalic acid or the di-p-toluoyl-L-(+)-tartaric acid in step-(b) is used directly or in the form of a solution of oxalic acid or di-p-toluoyl-L-(+)-tartaric acid dissolved in a solvent selected from the group consisting of water, an ether, an alcohol, a ketone, a chlorinated hydrocarbon, a hydrocarbon, an ester, a nitrile, and mixtures thereof.

Combining of the first solution with acid in step-(b) is done in a suitable order, for example, the first solution is added to the acid, or alternatively, the acid is added to the first solution. The addition is, for example, carried out drop wise or in one portion or in more than one portion. The addition is specifically carried out at a temperature of below about 50° C. for at least 15 minutes and more specifically at about 5° C. to about 35° C. for about 30 minutes to about 2 hours. After completion of addition process, the resulting mass is specifically stirred at a temperature of below about 50° C. for at least 30 minutes and more specifically at a temperature of about 20° C. to about 50° C. for about 1 hour to 10 hours to produce a second solution or suspension.

The second solution or suspension obtained in step-(b) is optionally heated at a temperature of about 40° C. to about 80° C. for at least 20 minutes and more specifically at a temperature of about 40° C. to about 75° C. for about 30 minutes to about 4 hours.

The isolation of pure solid state form of unsaturated cinacalcet salt in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In one embodiment, the crystallization is carried out by cooling the solution at a temperature of below 30° C. for at least 15 minutes, specifically at about 0° C. to about 25° C. for about 30 minutes to about 20 hours.

The recovering in step-(c) is carried out by methods such as filtration, filtration under vacuum, decantation, centrifugation, or a combination thereof. In one embodiment, solid state form of unsaturated cinacalcet salt is recovered by filtration employing a filtration media of, for example, a silica gel or celite.

The substantially pure solid state form of unsaturated cinacalcet salt obtained by above process may be further dried by the methods as described above.

Cinacalcet or a pharmaceutically acceptable salt thereof, preferably cinacalcet hydrochloride, can be prepared in high purity by using the pure unsaturated cinacalcet hydrochloride, oxalate and di-p-toluoyl-L-(+)-tartrate salts obtained by the process disclosed herein, by the methods disclosed hereinabove.

### Instrumental Details:

### X-Ray Powder Diffraction (P-XRD):

The X-Ray powder diffraction was measured by an X-ray powder diffractometer equipped with a Cu-anode (λ=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2θ; step width=0.01579°; and measuring time per step=0.11 second.

### Infra Red (FT-IR) Spectroscopy:

FT-IR spectroscopy was measured out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

The following examples are given for the purpose of illustrating the present invention and should not be considered as limitation on the scope or spirit of the invention.

### EXAMPLES

### Example 1

### Preparation of (R)-α-Methyl-N-[3-[3-(Trifluoromethyl)Phenyl]Propylene]-1-Naphthalenemethane amine hydrochloride salt (Unsaturated Cinacalcet hydrochloride salt)

Step-I: Preparation of Unsaturated Cinacalcet free base
(R)-(+)-1-(1-Naphthyl)ethyl amine (47.0 g) was added to a solution of 3-trifluoromethyl cinnamaldehyde (50.0 g) in methanol (250 mL), and the reaction mass was stirred at 25-30° C. for 4 hours. Sodium borohydride (9.45 g) was added portion wise to the reaction mass over a period of 1 hour and the reaction mixture was stirred at 25-30° C. for 4 hours. The reaction mass was cooled to 5-10° C. followed by slow addition of water (100 mL) and the pH of the resulting mass was adjusted to 2.0 with 10% HCl. The product was extracted with ethyl acetate (300 mL) and washed thrice with water (200 mL). The organic layer was concentrated under vacuum at below 50° C. to afford the unsaturated base.
Step-II: Preparation of Unsaturated Cinacalcet hydrochloride
The crude base obtained in step-I was dissolved in acetonitrile (150 ml), and concentrated hydrochloric acid (25.2 mL) was added drop wise slowly at 5-10° C. for 30 minutes. The reaction mixture was stirred at 25-30° C. for 3 hours followed by cooling the mass to 0-5° C. and stirred for 1 hour at 0-5° C. The resulting compound was filtered and washed with chilled acetonitrile (150 mL) and then dried the product at 50-60° C. to produce 55 g of the product. The product was further purified by recrystallization from acetonitrile to give 46 g of unsaturated cinacalcet hydrochloride (Purity by HPLC: 97.0%)

### Example 2

### Preparation of (R)-α-Methyl-N-[3-[3-(Trifluoromethyl)Phenyl]Propylene]-1-Naphthalenemethaneamine oxalate salt (Unsaturated Cinacalcet oxalate salt)

Crude unsaturated cinacalcet free base (5 g, obtained in step-I of example I) was dissolved in acetonitrile (40 mL) and a solution of oxalic acid (3.9 g, 1.2 mole) in acetonitrile (40 mL) was added at 25-30° C. The stirring was continued for 1-2 hours at 25-30° C. The precipitated salt was filtered and washed with chilled acetonitrile (20.0 mL). The wet material was dried at 50° C. to give 4.9 g of the unsaturated cinacalcet oxalate salt (Purity by HPLC: 98.29%).

### Example 3

### Preparation of Pure Unsaturated Cinacalcet Hydrochloride

Water (250 mL) was added to unsaturated cinacalcet oxalate (25 g, obtained in example 2) under stirring at 25-30° C. followed by addition of 10% sodium hydroxide solution (100 mL) to adjust the pH of the reaction mixture up to 10. The reaction mixture was stirred for 1 hour at 25-30° C. followed by the addition of ethyl acetate (250 mL) and then stirred for 30 minutes at 25-30° C. The layers were separated and the aqueous layer was extracted with ethyl acetate (100 mL). The organic layers were combined and washed with brine solution (400 mL). The resulting organic layer was dried over sodium sulfate and evaporated under vacuum at 50° C. to get 18.0 g of unsaturated cincalcet free base. The base was dissolved in acetonitrile (90 mL) and concentrated hydrochloric acid (6.3 mL) was added drop wise for 30 minutes at 5-10° C. The reaction mixture was stirred for 3 hours at 25-30° C. The resulting mass was cooled to 0-5° C. and stirred for 1 hour at 0-5° C. The separated solid was filtered, washed with chilled acetonitrile (36 mL) and then dried the product at 50-60° C. to produce 13.0 g of the desired product (Yield: 63.0%). The obtained product was recrystallized in acetonitrile to afford 11 g of pure unsaturated cinacalcet hydrochloride (Yield: 85.0%; Purity by HPLC: 98.5%).

### Example 4

### Preparation of (R)-α-Methyl-N-[3-[3-(Trifluoromethyl)Phenyl]Propylene]-1-Naphthalenemethaneamine Di-p-toluoyl-L-tartaric acid salt (Unsaturated Cinacalcet Di-p-toluoyl-L-tartrate salt)

Crude unsaturated cinacalcet free base (40 g, obtained in step-I of example I) was dissolved in methanol (100 mL) followed by the addition of a solution of di-p-toluoyl-L-tartaric acid (34.2 g) in methanol (100 mL) under stirring at 25-30° C. The stirring was continued for 1-2 hours at ambient temperature. The precipitated product was filtered, washed with methanol (100 mL) and then dried at 50° C. to afford 47.3 g of unsaturated cinacalcet di-p-toluoyl-L-tartrate salt (Purity by HPLC: 95.29%).

### Example 5

### Preparation of Pure Unsaturated Cinacalcet Hydrochloride

Water (450 mL) was added to unsaturated cinacalcet di-p-toluoyl-L-tartrate salt (30 g, obtained in example 4) under stirring at 25-30° C. followed by addition of 10% sodium hydroxide solution (150 mL) to adjust the pH of the reaction mixture up to 10. The reaction mixture was stirred for 3 hours at 25-30° C. followed by the addition of ethyl acetate (300 mL) and stirred for 30 minutes at 25-30° C. The layers were separated and the aqueous layer was extracted with ethyl acetate (150 mL). The both organic layers were combined and washed with brine solution (600 mL). The resulting organic layer was dried over sodium sulfate and evaporated under vacuum at 50° C. to get 14.0 g of unsaturated cincalcet free base. The base was dissolved in acetonitrile (70 mL) followed by drop wise addition of concentrated hydrochloric acid (5 mL) for 30 minutes at 5-10° C. The reaction mixture was stirred for 3 hours at 25-30° C. The resulting mass was cooled to 0-5° C. and stirred for 1 hour at 0-5° C. The separated solid was filtered, washed with chilled acetonitrile (28.0 mL) and then dried the product at 50-60° C. to afford the desired product 12.0 g (Yield: 77.0%). The product obtained was recrystallized in acetonitrile to afford 10.2 g of pure unsaturated cinacalcet hydrochloride (Yield: 85.0%; Purity by HPLC: 98.0%).

### Example 6

### Preparation of (R)-α-Methyl-N-[3-[3-(Trifluoromethyl)Phenyl]Propyl]-1-Naphthalene-methaneamine hydrochloride (Cinacalcet hydrochloride)

Unsaturated cinacalcet hydrochloride salt (19 g) was dissolved in n-butanol (1000 mL) and hydrogenated with 20% wet palladium hydroxide (0.25 g) at 1.5 Kg/Cm² for 3 hours at 5-10° C. The catalyst was removed by filtration and the solvent was stripped off below 70° C. under vacuum. The resulting crude product was dissolved in n-butanol (70 mL) and heated to 70° C. to obtain clear solution. The resulting solution was cooled to 0-5° C. and maintained for 1 hour. The isolated compound was filtered, washed with chilled n-butanol and then dried at 70° C. under vacuum to give 10 g of the pure cinacalcet hydrochloride (Purity by HPLC: 99.5%).

### Example 7

### Preparation of (R)-α-Methyl-N-[3-[3-(Trifluoromethyl)Phenyl]Propyl]-1-Naphthalene methaneamine hydrochloride (Cinacalcet hydrochloride)

Unsaturated cinacalcet hydrochloride salt (23 g) was dissolved in methanol (100 mL) and hydrogenated with 20% wet palladium hydroxide (0.59 g) under pressure of 1.5 Kg/Cm² for 3 hours at 5-10° C. The catalyst was removed by filtration and the solvent was stripped off at 70° C. under vacuum. The resultant crude product was dissolved in n-butanol (88 mL) and heated to 70° C. to obtain clear solution. The resulting solution was cooled to 0-5° C. and maintained for 1 hour. The isolated compound was filtered, washed with chilled n-butanol and then dried at 70° C. under vacuum to afford 11 g of the pure cinacalcet hydrochloride (Purity by HPLC: 99.71%).

### Example 8

### Preparation of (R)-α-Methyl-N-[3-[3-(Trifluoromethyl)Phenyl]Propyl]-1-Naphthalene-methaneamine hydrochloride (Cinacalcet hydrochloride)

Unsaturated cinacalcet hydrochloride (25 g) was dissolved in ethyl acetate (300 mL). Water (100 mL) was added to the above solution and basified with 25% aqueous sodium carbonate solution (50 mL). The resulting organic layer was separated out and charged into an autoclave vessel. 20% wet palladium hydroxide (0.62 g) was added to the above solution and hydrogenated for 3 hours at 5-10° C. under pressure of 1.5 Kg/Cm². After completion of the reaction, the catalyst was removed by filtration and the solvent was stripped off at 50° C. under vacuum. The obtained crude cinacalcet free base was dissolved in tetrahydrofuran (50 mL) followed by the addition of a solution of sodium bicarbonate (9.8 g) in water (100 mL). BOC anhydride (14.1 g) was added to the resultant reaction mixture at 10-15° C. and stirred at 25-30° C. for overnight. After completion of the reaction, the reaction mass was quenched with water (100 mL) and extracted with ethyl acetate (100.0 mL). The resulting organic layer was washed twice with water (100 mL) and solvent was evaporated under vacuum at below 60° C. to get N—BOC protected cinacalcet base. Concentrated hydrochloric acid (8.5 mL) was added to the solution of N—BOC protected cinacalcet dissolved in methanol (100 mL) and refluxed for 3 hours. Water (200 mL) was added to the reaction mixture drop wise at 60° C. for 1 hour. The reaction mass was allowed to cool at 25-30° C. and stirred 4 hours. The separated compound was filtered, washed with 50% aqueous methanol (100 mL) and then dried under vacuum at 60° C. to afford cinacalcet hydrochloride in pure form (19.0 g, Purity by HPLC: 99.8%).

### Example 9

### Preparation of (R)-α-Methyl-N-[3-[3-(Trifluoromethyl)Phenyl]Propyl]-1-Naphthalenemethaneamine hydrochloride (Cinacalcet hydrochloride)

Unsaturated cinacalcet hydrochloride (25 g) was dissolved in ethyl acetate (300 mL). Water (100 mL) was added to the above solution and basified with 25% aqueous sodium carbonate solution (50 mL). This was followed by the evaporation of solvent under vacuum at 50° C. and the crude product was dissolved in tetrahydrofuran (100 mL). Sodium bicarbonate (9.8 g) dissolved in water (100 mL) was added to the above solution followed by the addition of BOC anhydride (14.1 g) at 10-15° C. The reaction mixture was maintained overnight at ambient temperature. After completion of the reaction, water (100 mL) was added to the reaction mass and extracted twice with ethyl acetate (100 mL). The resulting organic layer was washed twice with water (100 mL) and dried over sodium sulfate. The organic layer was concentrated under vacuum at 60° C. to afford 23 g of N—BOC protected unsaturated cinacalcet. The resultant crude product was dissolved in methanol (1000 mL) and hydrogenated with 20% wet palladium hydroxide (0.6 g) under pressure of 1.0 Kg/Cm² for 3 hours at 5-10° C. The catalyst was removed by filtration and evaporated under vacuum at 60° C. to yield N—BOC protected cinacalcet freebase. Concentrated HCl (8.5 mL) was added to a solution of the above crude N—BOC protected cinacalcet freebase dissolved in methanol (100 mL) and refluxed for 3 hours. This was followed by drop wise addition of water (200 mL) at 60° C. for 1 hour. The resulting mass was allowed to cool at 25-30° C. and stirred for 4 hours. The resulting compound was filtered, washed with 50% aqueous methanol (100 mL) and then dried at 60° C. under vacuum to afford cinacalcet hydrochloride (16.0 g, Purity by HPLC: 99.8%).

1. A process for the preparation of cinacalcet hydrochloride of formula I: 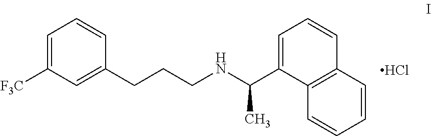
comprising hydrogenating (R)-α-methyl-N-[3-[3-(trifluoromethyl)phenyl]propylene]-1-naphthalenemethaneamine hydrochloride salt (unsaturated cinacalcet hydrochloride) of formula III: 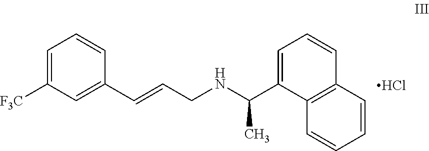
in the presence of a hydrogenation catalyst in a solvent to provide pure cinacalcet hydrochloride of formula I.

2. The process of claim 1, wherein the hydrogenation catalyst is selected from the group consisting of palladium hydroxide, palladium on carbon, platinum on carbon, platinum oxide, rhodium on carbon and rhodium on alumina; wherein the solvent used in the hydrogenation reaction is selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; and wherein the hydrogenation catalyst is used in the ratio of about 0.05% (w/w) to 10% (w/w) with respect to the unsaturated cinacalcet hydrochloride.

3-6. (canceled)

7. A process for the preparation of cinacalcet hydrochloride of formula I: 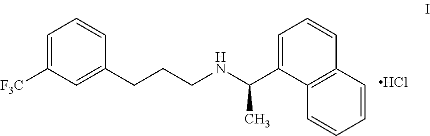
comprising:
a) reacting cinacalcet free base of formula II: 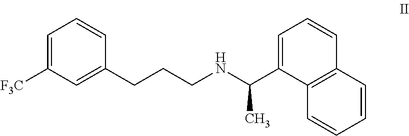
with a nitrogen protecting agent in the presence of a base in a first solvent to provide N-protected cinacalcet of formula IV: 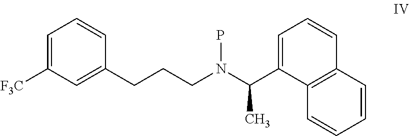
wherein ‘P’ represents a nitrogen protecting group; and
b) converting the compound of formula IV into pure cinacalcet hydrochloride of formula I by reaction with hydrochloric acid in a second solvent.

8. The process of claim 7, wherein the first and second solvents used in steps-(a) and (b) are, each independently, selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; wherein the base used in step-(a) is selected from the group consisting of triethyl amine, tributylamine, diisopropylethylamine, diethylamine, tert-butyl amine, N-methylmorpholine, pyridine, 4-(N,N-dimethylamino)pyridine, sodium hydroxide, calcium hydroxide, magnesium hydroxide, potassium hydroxide, lithium hydroxide, sodium carbonate, potassium carbonate, lithium carbonate, sodium bicarbonate and potassium bicarbonate; wherein the nitrogen protecting agent is an amine protecting agent selected from the group consisting of an acid anhydride, a mixed anhydride, an acid chloride, an alkyl halide, an aralkyl halide and a silyl compound; wherein the nitrogen protecting agent is used in a molar ratio of about 1 to 5 moles with respect to 1 mole of the cinacalcet free base; and wherein the nitrogen protecting group ‘P’ is selected from the group consisting of acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl.

9. The process of claim 8, wherein the first solvent is selected from the group consisting of water, tetrahydrofuran, dimethylformamide, dimethylsulfoxide, and mixtures thereof; wherein the second solvent is selected from the group consisting of water, methanol, ethanol, isopropyl alcohol, n-butanol, and mixtures thereof; wherein the nitrogen protecting agent is di-tert-butyl-dicarbonate; and wherein the nitrogen protecting group is tert-butoxycarbonyl (BOC).

10-16. (canceled)

17. An N-protected cinacalcet compound of formula IV: 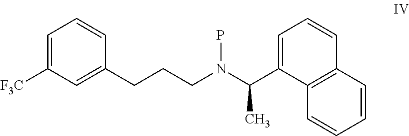
wherein ‘P’ represents a nitrogen protecting group selected from the group consisting of acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl.

18-20. (canceled)

21. A process for the preparation of cinacalcet hydrochloride of formula I: 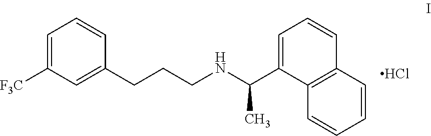
comprising:
a) neutralizing (R)-α-methyl-N-[3-[3-(trifluoromethyl)phenyl]propylene]-1-naphthalene methaneamine hydrochloride salt of formula III: 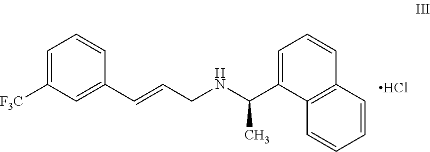
with a base in a first solvent to provide (R)-α-methyl-N-[3-[3-(trifluoromethyl)phenyl]propylene]-1-naphthalene methaneamine of formula V: 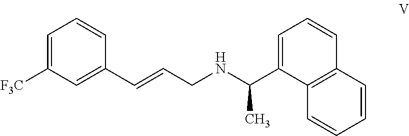
b) reacting the unsaturated compound of formula V with a nitrogen protecting agent in the presence of a base in a second solvent to provide N-protected unsaturated compound of formula VI: 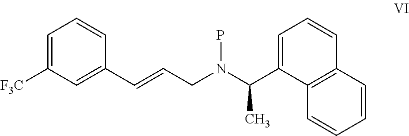
wherein ‘P’ represents a nitrogen protecting group;
c) hydrogenating the compound of formula VI in the presence of a hydrogenation catalyst in a third solvent to provide the N-protected cinacalcet of formula IV: 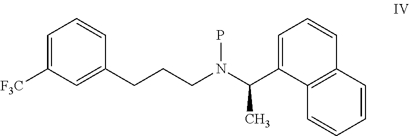
wherein P is as defined in formula VI;
d) converting the compound of formula IV into pure cinacalcet hydrochloride of formula I by reaction with hydrochloric acid in a fourth solvent.

22. The process of claim 21, wherein the first, second, third and fourth solvents used in steps-(a), (b), (c) and (d) are, each independently, selected from the group consisting of water, methanol, ethanol, isopropyl alcohol, propanol, t-butanol, n-butanol, acetone, methyl ethyl ketone, methyl isobutyl ketone, diethyl ketone, ethyl acetate, methyl acetate, isopropyl acetate, tert-butyl methyl acetate, ethyl formate, acetonitrile, tetrahydrofuran, dimethylformamide, dimethylsulfoxide, dioxane, diethyl carbonate, and mixtures thereof; wherein the base used in step-(a) or step-(b) is selected from the group consisting of triethyl amine, tributylamine, diisopropylethylamine, diethylamine, tert-butyl amine, N-methylmorpholine, pyridine, 4-(N,N-dimethylamino)pyridine, sodium hydroxide, calcium hydroxide, magnesium hydroxide, potassium hydroxide, lithium hydroxide, sodium carbonate, potassium carbonate, lithium carbonate, sodium bicarbonate and potassium bicarbonate; wherein the nitrogen protecting agent is an amine protecting agent selected from the group consisting of an acid anhydride, a mixed anhydride, an acid chloride, an alkyl halide, an aralkyl halide and a silyl compound; wherein the nitrogen protecting agent is used in a molar ratio of about 1 to 5 moles per 1 mole of (R)-α-methyl-N-[3-[3-(trifluoromethyl)phenyl]propylene]-1-naphthalene methaneamine of formula V; wherein the nitrogen protecting group ‘P’ is selected from the group consisting of acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl; and wherein the hydrogenation catalyst used in step-(c) is selected from the group consisting of palladium hydroxide, palladium on carbon, platinum on carbon, platinum oxide, rhodium on carbon, and rhodium on alumina.

23. The process of claim 22, wherein the first solvent is selected from the group consisting of water, ethyl acetate, and mixtures thereof; wherein the second solvent is selected from the group consisting of water, tetrahydrofuran, and mixtures thereof; wherein the third solvent is selected from the group consisting of methanol, n-butanol, and mixtures thereof; wherein the fourth solvent is selected from the group consisting of water, methanol, n-butanol, and mixtures thereof; wherein the nitrogen protecting agent is di-tert-butyl-dicarbonate; and wherein the nitrogen protecting group ‘P’ is tert-butoxycarbonyl (BOC).

24-31. (canceled)

32. An N-protected unsaturated cinacalcet compound of formula VI: 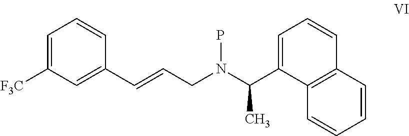
wherein ‘P’ represents a nitrogen protecting group selected from the group consisting of acetyl, pyrrolidinylmethyl, cumyl, benzhydryl, trityl, benzyloxycarbonyl (Cbz), 9-fluorenylmethyloxy carbonyl (Fmoc), benzyloxymethyl (BOM), pivaloyloxymethyl (POM), trichloroethxoycarbonyl (Troc), 1-adamantyloxycarbonyl (Adoc), allyl, allyloxycarbonyl, trimethylsilyl, tert.-butyldimethylsilyl, triethylsilyl (TES), triisopropylsilyl, trimethylsilylethoxymethyl (SEM), t-butoxycarbonyl (BOC), t-butyl, 1-methyl-1,1-dimethylbenzyl and pivaloyl.

33. The compound of claim 32, wherein the nitrogen protecting group ‘P’ is selected from the group consisting of acetyl, benzyloxycarbonyl (Cbz), trimethylsilyl, triethylsilyl (TES), trimethylsilyethoxymethyl (SEM), tert-butoxycarbonyl (BOC) and pivaloyl.

34. (canceled)

35. (canceled)

36. A salt of (R)-α-methyl-N-[3-[3-(trifluoromethyl)phenyl]propylene]-1-naphthalenemethane amine (unsaturated cinacalcet salt), wherein the salt of unsaturated cinacalcet is a hydrochloride salt, an oxalate salt or a di-p-toluoyl-L-(+)-tartrate salt.

37. The unsaturated cinacalcet salt of claim 36, which is in a solid state form.

38. (canceled)

39. (canceled)

40. The solid unsaturated cinacalcet salt of claim 37, having the following characteristics, wherein:
a) the solid state form of unsaturated cinacalcet hydrochloride salt is characterized by one or more of the following properties:
i) a powder X-ray diffraction pattern substantially in accordance with FIG. 1;
ii) a powder X-ray diffraction pattern having peaks at about 8.2, 14.3, 16.5, 19.5 and 21.9±0.2 degrees 2-theta;
iii) a powder X-ray diffraction pattern having additional peaks at about 10.5, 12.5, 13.1, 13.9, 14.7, 15.2, 15.8, 17.6, 20.6, 22.3, 23.1, 23.8, 24.3, 27.2 and 28.2±0.2 degrees 2-theta;
iv) an IR spectrum substantially in accordance with FIG. 2; and
v) an IR spectrum having absorption bands at 3427, 3049, 2935, 2906, 2775, 2705, 2473, 1597, 1586, 1454, 1432, 1384, 1329, 1274, 1255, 1200, 1173, 1163, 1127, 1071, 992, 957, 801, 781, 768 and 697±1 cm⁻¹;
b) the solid state form of unsaturated cinacalcet oxalate salt is characterized by one or more of the following properties:
i) a powder X-ray diffraction pattern substantially in accordance with FIG. 3;
ii) a powder X-ray diffraction pattern having peaks at about 6.3, 19.1 and 23.9±0.2 degrees 2-theta;
iii) a powder X-ray diffraction pattern having additional peaks at about 3.2, 12.7, 13.2, 14.1, 16.3, 16.9, 17.6, 20.2, 20.9, 21.3, 21.5, 23.1 and 25.6±0.2 degrees 2-theta;
iv) an IR spectrum substantially in accordance with FIG. 4; and
v) an IR spectrum having absorption bands at 3430, 3031, 2827, 1714, 1602, 1453, 1400, 1348, 1328, 1199, 1160, 1124, 1073, 906, 798, 777 and 703±1 cm⁻¹; and
c) the solid state form of unsaturated cinacalcet di-p-toluoyl-L-(+)-tartrate salt is characterized by one or more of the following properties:
i) a powder X-ray diffraction pattern substantially in accordance with FIG. 5;
ii) a powder X-ray diffraction pattern having peaks at about 4.6, 10.3, 11.1, 19.4, 20.3, 22.8 and 23.4±0.2 degrees 2-theta;
iii) a powder X-ray diffraction pattern having additional peaks at about 6.7, 6.9, 11.7, 12.3, 12.7, 13.9, 17.8, 18.4, 18.8, 20.0, 21.4, 22.3 and 25.6±0.2 degrees 2-theta;
iv) an IR spectrum substantially in accordance with FIG. 6; and
v) an IR spectrum having absorption bands at 3499, 3384, 2989, 2949, 2818, 2739, 2705, 1706, 1633, 1611, 1582, 1440, 1385, 1340, 1332, 1270, 1177, 1160, 1122, 1110, 1074, 970, 902, 777, 755 and 695±1 cm⁻¹.

41. A process for the preparation of unsaturated cinacalcet salt of claim 36, comprising:
a) providing a first solution of unsaturated cinacalcet free base in a solvent, wherein the solvent is selected from the group consisting of water, methyl tert-butyl ether, diethyl ether, diisopropyl ether, monoglyme, diglyme, tetrahydrofuran, dioxane, methanol, ethanol, n-propanol, isopropyl alcohol, isobutanol, n-butanol, tert-butanol, amyl alcohol, isoamyl alcohol, acetone, methyl ethyl ketone, methyl isobutyl ketone, methyl tert-butyl ketone, ethyl acetate, methyl acetate, isopropyl acetate, tert-butyl methyl acetate, ethyl formate, methylene chloride, ethylene dichloride, n-pentane, n-hexane, n-heptane and their isomers, cyclohexane, toluene, xylene, acetonitrile, and mixtures thereof;
b) combining the first solution with an acid to produce a second solution or suspension containing unsaturated cinacalcet acid addition salt, wherein the acid is selected from the group consisting of hydrochloric acid, oxalic acid and di-p-toluoyl-L-(+)-tartaric acid; and
c) isolating and/or recovering solid state form of unsaturated cinacalcet salt from the second solution or suspension.

42. (canceled)

43. The process of claim 41, wherein the solvent used in step-(a) is selected from the group consisting of water, methanol, ethanol, isopropyl alcohol, acetonitrile, and mixtures thereof.

44. The process of claim 41, wherein the first solution of unsaturated cinacalcet free base in step-(a) is prepared i) by dissolving unsaturated cinacalcet free base in the solvent at a temperature of 0° C. to the boiling temperature of the solvent; or ii) by reacting 3-(trifluoromethyl)cinnamaldehyde with (R)-(+)-1-(1-naphthyl)ethyl amine or an acid addition salt thereof in the presence of a reducing agent in a solvent to produce a reaction mass containing (R)-α-methyl-N-[3-[3-(trifluoromethyl)phenyl]propylene]-1-naphthalene methaneamine (unsaturated cinacalcet free base), subjecting the reaction mass to a filtration, a washing, an extraction, an evaporations, or a combination thereof, and dissolving or extracting the unsaturated cinacalcet free base in the solvent at a temperature of 0° C. to the boiling temperature of the solvent; or iii) by treating an acid addition salt of unsaturated cinacalcet with a base to produce unsaturated cinacalcet free base and dissolving or extracting the unsaturated cinacalcet free base in the solvent.

45. The process of claim 41, wherein the first solution obtained in step-(a) is optionally subjected to carbon treatment or silica gel treatment; wherein the combining in step-(b) is accomplished by adding the first solution to the acid or by adding the acid to the first solution; and wherein the second solution or suspension obtained in step-(b) is optionally heated at a temperature of about 40° C. to about 80° C. for at least 20 minutes.

46-50. (canceled)

51. The process of claim 41, wherein the isolation in step-(c) is carried out by cooling the solution at a temperature of about 0° C. to about 25° C. for about 30 minutes to about 20 hours.

52. The process of claim 41, wherein recovering in step-(c) is carried out by filtration, filtration under vacuum, decantation, centrifugation, filtration employing a filtration media of a silica gel or celite, or a combination thereof.

53. (canceled)

54. (canceled)
